(19) United States
(12) Patent Application Publication
(10) Pub. No.: US 2026/0317711 A1
(43) Pub. Date: Oct. 15, 2026

(54) Generation of highly potent Vd2+ T cell product from human peripheral blood

(71) Applicant: NantBio, Inc., Culver City, CA (US)

(72) Inventors: Zilei Liu, San Diego, CA (US); Syed Raza Ali, San Diego, CA (US); Manju Saxena, San Diego, CA (US); Leonard Sender, San Diego, CA (US); Patrick Soon-Shiong, Culver City, CA (US)

(73) Assignee: NantBio, Inc., Culver City, CA (US)

(21) Appl. No.: 19/647,531

(22) Filed: Apr. 14, 2026

Related U.S. Application Data
(60) Provisional application No. 63/789,359, filed on Apr. 15, 2025.
(60) Provisional application No. 63/840,377, filed on Jul. 8, 2025.

(51) Int. Cl. C12N 5/0783; A61K 31/675; A61K 35/14; A61K 38/20; C07K 14/57; C12N 5/00

(52) U.S. Cl. CPC C12N 5/0638; A61K 31/675; A61K 35/14; A61K 38/2086; C07K 14/57; C12N 5/0087; C12N 2501/2312; C12N 2501/2315; C12N 2501/2318

(57) ABSTRACT

Vδ2 T cells with increased cytotoxicity are prepared from apheresis material, or an αβ depleted T cell fraction and/or or a CD4/CD8 depleted T cell fraction using zoledronic acid and IL-15. Advantageously, the preparations presented herein do not require a 2-stage expansion and yield a cell preparation with a superior cytotoxicity profile, particularly where the cells are obtained from an αβ depleted T cell fraction and/or a CD4/CD8 depleted T cell fraction.

This application claims priority to U.S. provisional patent applications with the serial numbers 63/789,359 (filed Apr. 15, 2025) and 63/840,377 (filed July 8, 2025), each of which is incorporated by reference herein.

### FIELD OF THE INVENTION

The field of the invention is generation of T cells for therapy, and especially as it relates to culture/expansion and activation of Vδ2 T cells.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Within the relatively large number of various cell types making up the immune system, γδ T cells are a small subset of T lymphocytes in adult peripheral blood, typically constituting 1% to 10% of CD3⁺ T cells. Unlike conventional T cells that rely on antigen-derived peptides presented by human leukocyte antigen (HLA), γδ T cells engage their ligands independently of HLA restriction, placing them at the intersection of innate and adaptive immunity. Vδ2 T cells, which represent 50%-90% of γδ T cells, are abundant in peripheral blood. Vδ2 T cells can directly recognize and eliminate multiple tumor targets by recognizing phosphate antigens (pAgs). As such, Vδ2 T cells have become an attractive option for use of these cells as a therapeutic.

To that end, various protocols for ex vivo expansion of Vδ2 T cells from human blood have been developed, including zoledronic acid-based approaches used in clinical settings. Zoledronic acid, a medication for bone diseases, inhibits farnesyl pyrophosphate synthase, leading to the accumulation of isopentenyl pyrophosphate (IPP) on antigen-presenting cells (APCs) and tumor cells, activating Vδ2 T cells. Following activation, these T cells are cultured with high concentrations of IL-2 (500-1000 IU/mL) for clinical-scale expansion. In the established protocol, PBMCs are treated with zoledronic acid, supplied with high concentration of IL-2(500 -1000 IU/mL). After 7 to 10 days of expansion, αβ T cell depletion is performed to remove remaining αβ T cells, followed by a second phase of expansion supplied with IL-2 to yield high purity of Vδ2 T cells.

In some cases of manufacture, αβ T cell depletion can be performed before harvesting. However, some reports indicate the failure of expanding Vδ2 T cells from αβ T cell depleted cell fraction. For example, in one approach developed by Cytomed Therapeutics, PBMCs are treated with Zoledronic acid to initiate the 1st phase of activation and expansion. After αβ T cell depletion in the middle of expansion, irradiated CD64, CD86 and CD137L-expressing K562 cells were used as APCs to further expand Vδ2 T cells in combination with anti-CD3 antibody (Clone: OKT3). While this method achieves >200,000-fold expansion in 18-21 days, the use of leukemia K562 feeder cells raise safety concerns for clinical applications.

Once isolated, Vδ2 T cells can be further selected for heightened cytotoxicity and potent antibody-dependent cell-mediated cytotoxicity (ADCC) functionality using CD16 as a marker (see e.g., Nature Communications (2023) 14:6942). In other attempts to enhance cytotoxicity, Vδ2 T cells were exposed to TGF-β (see e.g., Oncoimmunology 2019, VOL. 8, NO. 1, e1522471). However, while conceptually attractive, subsequent isolation or further exposure with additional agents will increase complexity and risk of contamination. Further known methods generally following the processes noted above include those described in U.S. Pat. No. 7,749,760, US 2019/0175650, US 2017/0296584, and US 2023/0323298.

Thus, even though various systems and methods of preparing Vδ2 T cells for therapeutic use are known in the art, all or almost all of them suffer from several drawbacks. Therefore, there remains a need for improved systems and methods for simple and effective preparation of Vδ2 T cells, especially where such cells are desired for therapeutic use with improved cytotoxicity.

### SUMMARY OF THE INVENTION

The inventive subject matter is directed to culture and expansion of Vδ2 T cells to so produce therapeutic compositions containing such cells, where the so produced cells are obtained in a protocol that does not require a 2-step process, and where the so produced cells have increased cytotoxicity and exhibit lower T cell exhaustion as compared to cells produced in a conventional method.

In one aspect of the inventive subject matter, the inventors contemplate a method of expanding Vδ2 T cells from human apheresis material that includes a step of at least partially depleting the apheresis material of αβ T cells and/or CD4/CD8 T cells to so form an at least partially depleted T cell fraction, a further step of exposing the at least partially depleted T cell fraction to Zoledronic acid and a stabilized IL-15:IL-15Rα for 10-14 days, and a still further step of stimulating expansion of memory-like Vδ2 T cells with a cytokine cocktail comprised of Interleukin 12 (IL-12), Interleukin 15 (IL-15), and Interleukin 18 (IL-18).

In some embodiments, the stabilized IL-15:IL-15Rα is N-803, which may be present at a concentration of 64-84 ng/ml. In further embodiments, the zoledronic acid may be present at a concentration of 5 μM. While not limiting the inventive subject matter, it is typically preferred that the cell media comprises X-VIVO™ 15 or T-VIVO®, and the medium may further include 5-10% human AB serum. Preferably, the cell media comprising the stabilized IL-15:IL-15Ra is replenished every 3-4 days, and/or where desired, the human apheresis material CD4/CD8 T cell depleted. In still further embodiments, the memory-like Vδ2 T cells are comprised of effector memory (Vδ2 Tem) and central memory (Vδ2 Tcm) T cells.

Preferably, but not necessarily, at least one of the Interleukin 12 (IL-12), Interleukin 15 (IL-15), and Interleukin 18 (IL-18) is a stabilized fusion protein derivative thereof, and the memory-like Vδ2 T cells are comprised of effector memory (Vδ2 Tem) T cells and/or the memory-like Vδ2 T cells have enhanced cytotoxicity against tumor cells. Moreover, it is contemplated that the IL-12 and IL-18 may be added to the cytokine cocktail as a fusion protein comprised thereof.

In yet further embodiments, the cytokine cocktail comprises N-803 in addition to or as replacement of IL-15, and/or the IL-12 and IL-18 are added to the cytokine cocktail as a fusion protein or fusion protein complex comprised thereof. In some embodiments, the cytokine cocktail is added after 10 days in culture. It is also contemplated that the Vδ2 T cells are enriched to comprise >90% of T cells after 10 days in culture.

In another aspect of the inventive subject matter, the inventors contemplate a pharmaceutical composition comprising human apheresis material, Zoledronic acid, and a stabilized IL-15:IL-15Rα. Preferably, but not necessarily, the human apheresis material is depleted of αβ T cells and/or CD4/CD8 T cells. As noted earlier, the stabilized IL-15:IL-15Rα may be N-803 (e.g., at a concentration of 64-84 ng/ml), and/or the zoledronic acid may be present at a concentration of 2-10 μM (for example 2-4 μM, 3-5 μM, 4-6 μM, 5-7 μM, 6-8 μM, 7-9 μM, 8-10 μM), and more preferably at a concentration of 5 μM. In further embodiments, it is also contemplated that the human apheresis material will be αβ T cell and/or CD4/CD8 T cell depleted.

Viewed from a different perspective, the inventors therefore also contemplate a kit comprising human apheresis material, zoledronic acid, and a stabilized IL-15:IL-15Rα, optionally in combination with directions for use thereof. Preferably, but not necessarily, the human apheresis material is depleted of αβ T cells and/or CD4/CD8 T cells. In still other contemplated examples, the stabilized IL-15:IL-15Rα may be N-803 (in an amount sufficient to enable a concentration of 64-84 ng/ml), and/or the zoledronic acid is present at an amount to enable a concentration range of 2-10 μM (for example 2-4 μM, 3-5 μM, 4-6 μM, 5-7 μM, 6-8 μM, 7-9 μM, 8-10 μM), and more preferably at a concentration of 5 μM.

Various objects, features, aspects, and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 illustrates exemplary flow plots showing Vδ2 T cell enrichment from (A) apheresis material and (B) αβ depleted T cell fraction.

FIG. 2 provides tables illustrating an exemplary comparison of the phenotypes of Zoledronic acid-activated Vδ2 T cells expanded with IL-2 or N-803 from the same donor.

FIG. 3 depicts an exemplary flow cytometric analysis showing N-803 supported generation of a Vδ2 T cell product containing mainly effector memory (Tem) and central memory (Tcm) subsets, which maintains cytotoxicity on tumor cells and proliferative potential.

FIG. 4 is a table summarizing exemplary expansion data of Vδ2 T cells from apheresis material and αβ T cell depleted fraction.

FIG. 5 shows exemplary tumor inhibition curves of expanded Vδ2 T cells at different E/T ratios on breast cancer SKBR3 and ovarian cancer SKOV3.

FIG. 6 illustrates an exemplary comparison of the phenotypes of expanded Vδ2 T cells with or without IL12/IL18/N-803 stimulation before harvesting.

FIG. 7 depicts exemplary results for tumor inhibition curves of unstimulated Vδ2 T cells and stimulated Vδ2 T cells (s-Vδ2 T) at different E/T ratios on breast cancer SKBr3 and ovarian cancer SKOV3.

FIG. 8A depicts exemplary results for tumor inhibition curves of unstimulated Vδ2 T cells and stimulated Vδ2 T cells (s-Vδ2 T) at various E/T ratios on ovarian cancer SKOV3 spheroid model on Day 17.

FIG. 8B depicts exemplary results for tumor inhibition curves of unstimulated Vδ2 T cells and stimulated Vδ2 T cells (s-Vδ2 T) at various E/T ratios on ovarian cancer SKOV3 spheroid model on Day 24.

FIG. 9 is a schematic illustration of manufacturing s-Vδ2 T cells based on CD4/CD8 depletion.

FIG. 10 depicts exemplary numerical results and summary of the development of Vδ2 /s-Vδ2 manufacturing process. Three independent studies were performed using apheresis material from different donors. Results showed the successful enrichment of Vδ2 T cells from 1-6% upon inoculation to >80% upon harvest day. And 3-7 billion cells were generated from 200-1000 million cells.

FIG. 11 depicts exemplary flow dot plot results for example of Vδ2 enrichment from apheresis to harvest based CD4/CD8 depletion process.

FIG. 12 depicts exemplary comparison of Vδ2+ and s-Vδ2+ T cell products. s-Vδ2+ T cells showed significantly stronger potency compared to Vδ2+ T cells. (A) Flow dot plots for the example of IFN-γ expression of Vδ2+ and s-Vδ2+ T cell harvests. s-Vδ2+ T cells showed >95% IFN-γ expression while Vδ2+ T cells only showed background signal. (B) Evaluation of cytotoxicity of Vδ2+ and s-Vδ2+ T cell on K562 using Calcein AM release assay. s-Vδ2+ showed significantly stronger potency in killing K562 target.

FIG. 13 depicts exemplary cytotoxicity results against K562 (CML), Raji (B cell lymphoma) and U266B1 (Multiple Myeloma) by Vδ2 and s-Vδ2 T cells obtained by the enrichment process of FIG. 9. s-Vδ2+ T cell products showed potent and stronger tumor cytotoxic capabilities on a range of tumor cell lines including CML (K562), B cell lymphoma (Raji) and Multiple Myeloma (U266B1) compared to Vδ2 T cells according to Calcein AM release assay.

FIG. 14 depicts exemplary tumor inhibition results against SKOV3-NLR, SKBR3-NLR, H441-NLR and H841-NLR cells (solid tumor cells) by Vδ2and s-Vδ2T cells obtained by the enrichment process of FIG. 9. s-Vδ2+ T cell products showed potent and stronger tumor inhibition on a range of solid tumor cell lines expressing red fluorescent protein (NLR) compared to Vδ2+ T cells. The tumor targets covered a wide range of clinical indications including Ovarian cancer, Breast cancer and lung cancer. Assay is performed on Incucyte.

FIG. 15 depicts exemplary tumor inhibition results against SKOV3-NLR spheroid model by Vδ2 and s-Vδ2 T cells obtained by the enrichment process of FIG. 9 in the presence of cytokines N-803, IL-12 or IL-15. s-Vδ2+ supplied with N-803 resulted in higher tumor inhibition compared to commonly used cytokines IL-2 and IL-15.

FIG. 16 depicts exemplary tumor rechallenge assay results against A549-NLR by Vδ2 and s-Vδ2 T cells obtained by the enrichment process of FIG. 9. Superior persistence of sustained antitumor potency of s-Vδ2+ T cells was observed. Table showed the IC50 of s-Vδ2+ and Vδ2+ against A549-NLR lung cancer cells based on Incucyte tumor inhibition assay.

FIG. 17 depicts exemplary data of Viability, cytotoxicity against K562 and intracellular IFN-γ expression of thawed Vδ2 and s-Vδ2 T cell drug products obtained by the process of FIG. 9.

### DETAILED DESCRIPTION

The inventors have now unexpectedly discovered that Vδ2 T cells with enhanced cytotoxicity can be prepared in a substantially simplified manner that does not require a 2-step enrichment process, that avoids IL-2 based T cell exhaustion, and that produces Vδ2 T cells that exhibit increased expression of CD56, CD25, and NKG2D as compared to a conventional process using zoledronic acid and IL-2. Such findings are particularly desirable as current clinically acceptable approaches involve a two-stage expansion in which αβ T cell depletion is required in the middle or the end of expansion, which increases the cost, variation and complexity of manufacturing. Moreover, currently known approaches often require high IL-2 concentrations.

More particularly, and as is described in more detail below, the inventors discovered that human apheresis material or an αβT cell depleted cell fraction from PBMCs (e.g., pre-processed by CliniMACS) can be seeded in a culture Flask with a cell medium (e.g., Xvivo15 or Tvivo) that is supplied with a defined amount of GMP-grade Zoledronic acid (Zol) and N-803 on Day 1 (inoculation). Cell media Xvivo15 or Tvivo supplied with defined amount of N803 can then be replenished to the cell culture at desired intervals (e.g., every 3-4 days). High purity of Vδ2 T cells (e.g., at least 90%, or at least 95%, or at least 97% purity) can then be harvested on expansion day 10 to day 14.

In most embodiments, N-803 may comprise an IL-15N72D:IL-15RαSu/Fc complex formulated for administration to a subject, and the N-803 complex may include a dimeric IL-15RαSu/Fc and two IL-15N72D molecules. In further embodiments, the IL-15N72D component may be a mutant form of human IL-15 with an asparagine to aspartic acid substitution at position 72. This mutation may enhance the biological activity of IL-15. The IL-15RαSu component may be a soluble form of the IL-15 receptor alpha chain. The Fc portion may be derived from human IgG1. Most typically, The N-803 complex may have a molecular weight of approximately 114 kDa and will advantageously exhibit an extended serum half-life compared to native IL-15 due to the Fc fusion. Among further benefits, it should also be noted that N-803 may stimulate proliferation and activation of NK cells and memory CD8+ T cells without stimulating Treg cells. Moreover, N-803 will also induce production of cytokines like IFN-γ. Therefore, N-803 and variants thereof may enhance antigen-specific T cell responses when combined with vaccines. Suitable variants of N-803 may include complexes with different IL-15 mutations, such as IL-15N72A. The Fc portion may be derived from other IgG subtypes. Linker sequences between the components may be modified. The ratio of IL-15 to IL-15Rα may be altered. Exemplary sequences for suitable N-803 and analogs are shown in U.S. patent application Ser. No. 19/557,689, which is incorporated by reference, and which forms express part of this disclosure.

Thusly cultivated cells can be further enhanced in potency by exposure to an IL12/IL18/N-803 stimulation cocktail (typically before harvest between day 10-14) for a defined time. As will be readily appreciated, the so activity enhanced cells can be washed prior to further modification, cryopreservation, or administration to a subject. In this context, it should be particularly appreciated that Vδ2 T cells can be prepared in therapeutically meaningful quantities in a single culture vessel and without any intervening purification or isolation step.

Moreover, it should also be noted that by the replacement of IL-2 with N-803, the cells isolated by the methods presented herein will unexpectedly not only allow for a substantially simplified process, but also exhibit higher levels of CD56, higher levels of CD25 (activation status), and higher levels of NKG2D (NK functional marker). Advantageously, the Vδ2 T cells produced by the methods presented herein will comprise predominantly effector memory (Tem) and central memory (Tcm) subsets with minimal terminal differentiated subset (Temra). Viewed from a different perspective, it should therefore be recognized that the methods presented herein can result in an expansion and enrichment of γδ T cells for clinical dosing. For example, and as demonstrated below, a 2,000-fold expansion of Vδ2 T cells was observed from an apheresis product in about 10-16 days while a 600-fold expansion of Vδ2 T cells was observed from ab T cell depleted fraction of PBMCs in about 10-16 days.

Beneficially, cells produced by the methods presented herein exhibited significant cytotoxicity against various cancer cells (e.g., breast cancer, ovarian cancer) at favorable effector to target cell ratios. Moreover, and as is also shown in more detail below, cytotoxicity of the so produced cells could be further enhanced by exposure to a cytokine cocktail comprising N-803, IL-12, and IL-18. Such exposure led to a significant increase of T cell activation markers CD69 and CD25, along with an increased expression of NK cell activation marker NKp30. Remarkably, the so stimulated Vδ2 T cells had even further increased killing activity against cancer cells and cancer cell spheroids compared to unstimulated Vδ2 T cells.

As will be readily appreciated, it is contemplated that the source of the apheresis material may vary considerably. However, especially preferred sources will be a fresh preparation from a donor (e.g., human HLA matched, human allogeneic, non-human HLA-engineered, etc.). In still further contemplated aspects, the apheresis material may be at least partially depleted of αβ T cells, typically using conventional separation techniques (e.g., using CliniMACS PLUS or CliniMACS Prodigy). Alternatively, the apheresis product, which may have been previously subjected to αβ T cell and/or CD4/CD8 depletion, may also be a cryopreserved product from an allogenic source or an HLA matched donor or may be an autologous product.

Additionally, it is contemplated that the medium for the culture/expansion of the Vδ2 T cells may vary considerably. However, it is typically preferred that the medium is a defined serum-free medium formulated to support growth and expansion of T cells such as Xvivo15 or Tvivo (Lonza Bioscience). As desired or needed, the medium may be further supplemented with AB serum (e.g., between 2% and 15% AB serum). Zoledronic acid is then supplied to the medium, typically in an amount of between 1-10 μM, most typically 5 μM, while N-803 will be added to a final concentration of between 30-100 ng/ml, such as between 64-84 ng/ml.

Of course, it should be noted that while N-803 is the preferred form of a stabilized IL-15:IL-15Rα, various other forms are also deemed suitable for use herein. For example, the IL-15 portion may be a native form of IL-15 or a superagonist mutant (e.g., N72D). In further examples, the IL-15 may also be extended to include further stimulatory functions, or truncated. Similarly, the IL-15 receptor α portion may be mutated to achieve higher binding affinity to IL-15 or to achieve higher affinity to another (α or γ) receptor chain, and/or may comprise a dimeric IL-15Rα sushi domain-IgG1 Fc fusion protein. Likewise, zoledronic acid may be at least partially substitute with another (amino)bisphosphonate.

Most typically, zoledronic acid may be used only on day 1 of the culture/expansion and need not be supplemented in subsequent media replenishment steps. However, in alternative embodiments, zoledronic acid exposure may be continued to at least day 4, or at least day 8, or at least day 10 of the culture/expansion (typically at the same or decreasing concentrations). On the other hand, it is generally preferred that the N-803 will be present throughout the culture and expansion, preferably at the same concentration as the initial concentration. In still further contemplated aspects, the time period for cultivation/expansion will typically be in the range of between 10-16 days, and further stimulation for enhanced cytotoxicity will preferably be performed in the same medium at or near the end of the 10-16 day period. Moreover, it should be noted that the entire culture/expansion and even stimulation protocol can be performed in a single bioreactor or culture flask, preferably without the need to have intermediate or final separation steps for one or more cell components. As such, the risk of contamination is substantially reduced and workflow simplified.

While not wishing to be bound by any theory or hypothesis, the inventors contemplate that the unexpected effects of N-803 vis-à-vis IL-2 are at least in part due to the increased persistence of N-803 (and with that signaling duration) as well as to the activation of distinct pathways. In addition, the binding of the Fc portion of N-803 to CD16 on the T cells may further contribute to the increased activation profile.

With respect to the further stimulation of the Vδ2 T cells to form s-Vδ2 T cells it is contemplated that the stimulation will be achieved by exposure of the Vδ2 T cells to a cocktail of IL12, IL18, and IL15 (or IL15 analogs such as N-803) over a period of no more than 48 hours, or no more than 36 hours, or no more than 24 hours, or no more than 18 hours, or no more than 12 hours, but typically more than 1 hour, or more than 2 hours, or more than 4 hours, or more than 8 hours. Most typically, further stimulation will use about 10 ng/mL IL-12, about 50 ng/mL IL-18, and about 100 ng/mL IL-15 or between 30-100 ng/ml N-803. However, other quantities are also deemed suitable, and appropriate quantities will be readily determined by quantification of their enhanced cytotoxicity and/or increased expression of CD69, CD25, and/or NKp30.

Once expanded or expanded and stimulated, it should be appreciated that the so produced cells can be formulated into various media suitable for therapeutic, diagnostic, or research use. Therefore, suitable formulations include various formulations appropriate for infusion, for cryopreservation, and for toxicity assays. For example, cryopreservation media will generally be cell-specific, optimized freeze media, which are designed to prepare and preserve cells in very low temperature environments (e.g., −70 °C to −120 °C). Furthermore, the cryopreservation medium is contemplated to provide a safe, protective environment during the freezing, storage, and thawing process for cells and tissues, and provide for enhanced cell viability and functionality while eliminating the need for serum, proteins or high levels of cytotoxic agents. Therefore, in one preferred embodiment, the cryopreservation medium may be CryoStor CS10, optionally containing albumin. Furthermore, the cryopreservation medium may be formulated such that it favors antibody binding to the cells presented herein.

In another example, the cell medium will be a pharmaceutically acceptable medium for infusion and will contain the cells presented herein in a therapeutically effective amount. The precise therapeutically effective amount is that amount of the composition that will yield the most effective results in terms of efficacy of cancer treatment in a given subject. This amount may vary depending upon a variety of factors, including but not limited to the characteristics of the cells, antibodies that may or may not be present, media compositions, etc., as well as the physiological condition of the subject (including age, sex, disease type and stage, general physical condition, responsiveness to a given dosage, and type of medication). One skilled in the clinical and pharmacological arts will be able to determine a therapeutically effective amount through routine experimentation, for instance, by monitoring a subject's response to administration of the pharmaceutical composition and adjusting the dosage accordingly. In a preferred embodiment, it is contemplated that the Vδ2 T cells will be administered at a dosage of between 5×10⁵ cells/kg and 5×10⁸ cells/kg.

Where desired, contemplated cells and compositions may further comprise an antibody that may be co-administered with the Vδ2 T cells. Therefore, in some embodiments the antibody may be injected or infused separately or contemporaneously. However, in other embodiments it is contemplated that the antibodies will be bound to CD of the Vδ2 T cells. As will be readily appreciated, the CD16 may be a natively expressed CD16 or a recombinantly expressed CD16 or other Fc receptor protein such as CD32 or CD64.

In still further aspects of the inventive subject matter, it is contemplated that the so prepared Vδ2 T cells or s-Vδ2 T cells may be further subjected to genetic engineering to express a chimeric antibody receptor (CAR). In such case, the introduction of the recombinant nucleic acid may be done prior to expansion, at an intermediate stage, or after expansion (optionally before stimulation). For example, CAR-engineering of Vδ2 T cells may use mRNA transduction. Here, mRNA encoding a CAR is electroporated into Vδ2 /s-Vδ2 T cells for CAR expression. After transduction, CAR expressions can then be measured using flow cytometry. And potencies assays using suitable targets can be developed to evaluate the efficacy of CAR-T products. In another example, lentiviral transfection is employed to engineer Vδ2 T cells to generate CAR-T products using. This method will generate Vδ2 T cell products stably expressing CARs. As will be readily appreciated, the type of CAR and target for the CAR may vary considerably, and all known CARs with all known targets are deemed suitable for use herein. However, particularly contemplated CARs will include an Fc-epsilon CAR, which may target cancer-associated antigens such as those described in WO 2019/226708, incorporated by reference in its entirety herein. In still further contemplated aspects, the cells presented herein may also recombinantly express a TGF-beta trap.

Therefore, the inventors particularly contemplate pharmaceutical composition and kits comprising the cells presented herein, especially in the treatment of solid and liquid cancers. Among other cancers, especially contemplated cancers include melanoma, breast cancer, ovarian cancer, gastric cancer, prostate cancer, squamous cell carcinoma, head and neck cancer, colon cancer, pancreatic cancer, uterine cancer, renal cell cancer, glioblastoma, medulloblastoma, sarcoma, and lung cancer.

### EXAMPLES

### Protocol 1

The following description illustrates a brief overview of one exemplary protocol for Vδ2 T cell enrichment/expansion where the Vδ2 T cells were prepared from an αβ depleted T cell fraction. In particular: Apheresis material as such or αβ depleted T cell fraction from CliniMACS PLUS or CliniMACS Prodigy were inoculated in G-Rex vessel at a VCD of 1-4×10⁶ cells/mL in X-VIVO® 15 or T-VIVO® media supplied with Zoledronic acid (5 mM) and N-803 (74 ng/mL). Fresh media containing N-803 were topped-up every 3-4 days. Here, zoledronic acid was employed only on day 1. Note that enriched and expanded cells can be inoculated in suitable size Xuri bioreactor for further expansion. Vδ2 T cell enrichment was monitored using a Flow cytometry-based assay. Notably, 90% Vδ2 T cell enrichment was observed by day 10. Enriched and expanded cells are then activated with cytokine cocktail containing N-803, IL-12 and IL-18 to produce stimulated Vδ2 T cells (memory). Stimulated Vδ2 T cells are more potent (i.e., have higher cytotoxicity) and were harvested and formulated in CS-10+HA for cryo-storage.

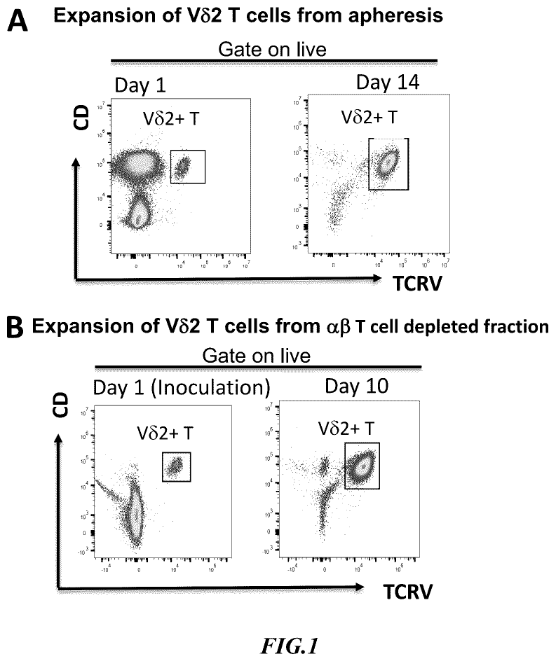

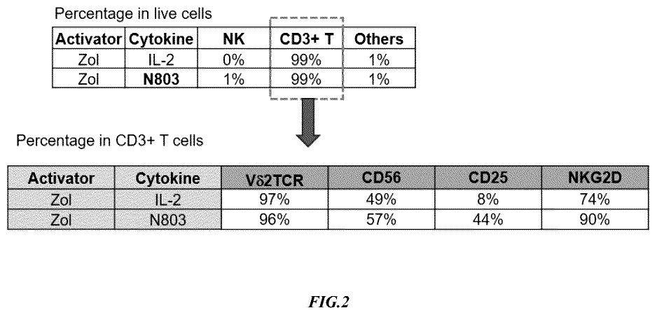

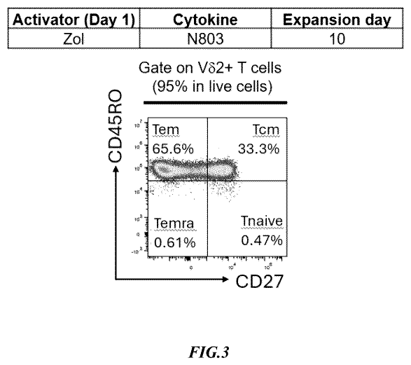

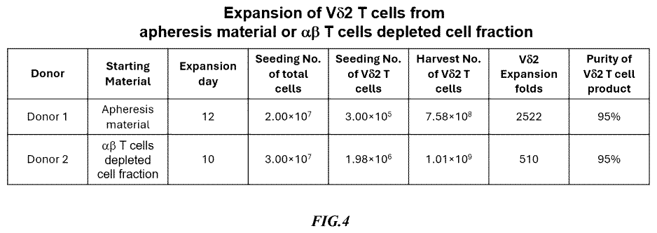

FIG. 1 illustrates exemplary flow plots showing Vδ2 T cell enrichment from apheresis material (A) and (B) αβ T cell depleted cell fraction. As can be readily seen, enrichment of the Vδ2 T cells was significant after 14 days of continuous culture from both starting materials: raw apheresis product and a αβ T cell depleted cell fraction. FIG. 2 shows tables illustrating an exemplary comparison of the phenotypes of Zol-activated Vδ2 T cells expanded with IL-2 or N-803 from the same donor. Remarkably, while both methods produced a high fraction of CD3+ T cells with Vδ2 T cell receptor, cells expanded with N-803 exhibited dramatically increased quantities of CD56, CD25, and NKG2D, indicating a substantially higher potential for cytotoxicity and activation. Results for the subtype analysis of the expanded cells are shown in FIG. 3 where flow cytometric analysis shows that N-803 generated a Vδ2 T cell product containing mainly effector memory (Tem) and central memory (Tcm) subsets, which advantageously maintain cytotoxicity on tumor cells and proliferative potential. FIG. 4 is an expansion summary of Vδ2 T cells from apheresis material and αβ T cell depleted fraction. Here it can be seen that expansion is not only substantial, but that the protocol produced therapeutically effective cells with desirable cytotoxicity profile starting from raw apheresis material as well as from an αβ T cell depleted cell fraction.

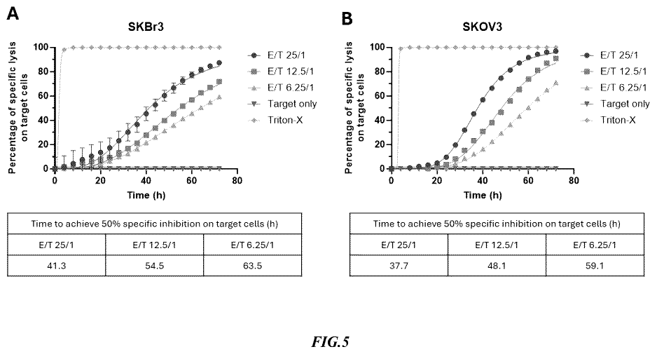

To investigate whether the so produced cells had cytotoxicity, the inventors tested the so produced cells against a breast cancer cell line SKBR3 and an ovarian cancer cell line SKOV3. Here, the assay was performed on an Incucyte Live Cell analysis platform and data were generated from two replicates. FIG. 5 depicts exemplary tumor inhibition curves of expanded Vδ2 T cells at different E/T ratios on breast cancer SKBR3 and ovarian cancer SKOV3. As can be readily taken from the graphs, the cells had substantial cytotoxicity against the cancer cell lines at a variety of effector to target ratios.

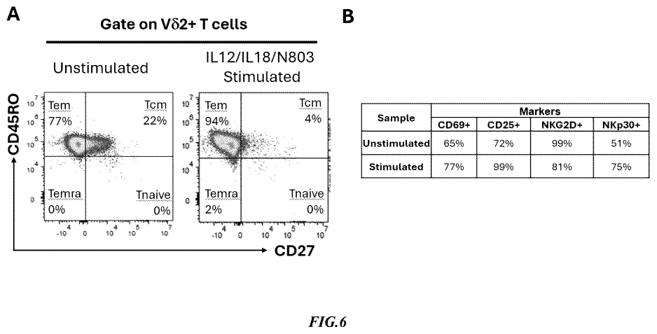

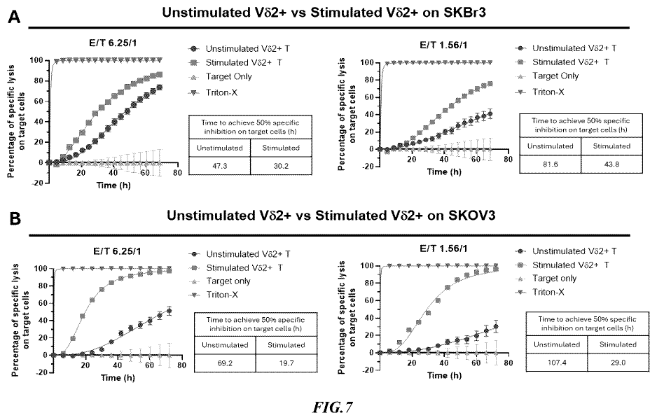

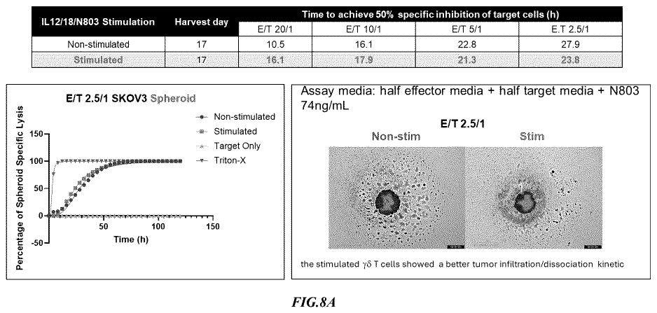

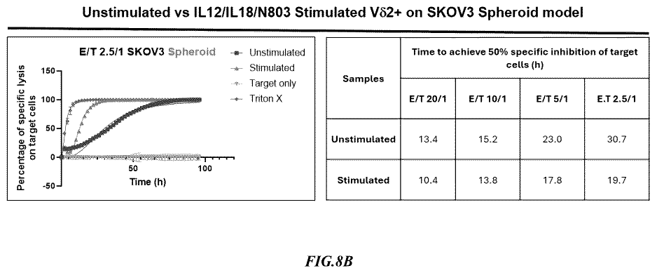

Further stimulation with a cytokine cocktail (here: IL12/IL-18/N-803) was performed on the cells and exemplary results are shown in the comparative graphs in FIG. 6. The data shown here illustrates the comparison of the phenotypes of expanded Vδ2 T cells with or without IL12/IL18/N-803 stimulation before harvesting. Notably, CD69, CD25, and NKp30 exhibited substantial increases, with the vast majority having an effector memory phenotype. The cytotoxic effect of the stimulation was then tested on breast cancer cell line SKBR3 and ovarian cancer cell line SKOV3. FIG. 7 shows the corresponding tumor inhibition curves of unstimulated Vδ2 T cells and stimulated Vδ2 T cells (s-Vδ2 T cells) at different E/T ratios on the tested cell lines. Here again the assay was performed on Incucyte Live Cell analysis platform and ten data were generated from three replicates. Finally, the inventors also tested unstimulated Vδ2 T cells and stimulated Vδ2 T cells (s-Vδ2 T cells) on a 3D ovarian cancer SKOV3 spheroid model. As can be seen from the graph and data in FIG. 8A and FIG. 8B, tumor inhibition increased significantly for stimulated Vδ2 T cells (s-Vδ2 T cells) against unstimulated Vδ2 T cells at E/T ratio of 2.5/1 on the ovarian cancer SKOV3 spheroid model. Assay was performed on Incucyte Live Cell analysis platform. Data was generated from three replicates.

### Protocol 2

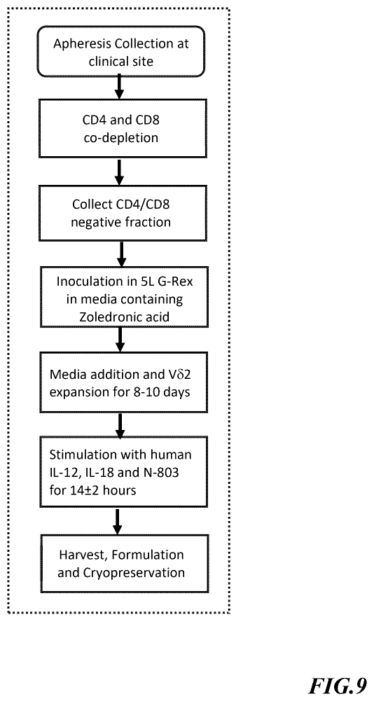

The following description illustrates a brief overview of an alternative exemplary protocol for Vδ2 T cell enrichment/expansion where the Vδ2 T cells were prepared from an CD4 and CD8 depleted T cell fraction or apheresis material. In particular: Apheresis material was subjected on Day 1 to CD4/CD8 depletion (e.g., using Cell Separation System to yield CD4/CD8 depleted cell fraction with enriched Vδ2 T cells). Then cells were processed by automated buffer exchange platform CTS Rotea or manually suspended in inoculation media containing Zoledronic acid (5 mM) and N-803 (74 ng/mL). Cells were subsequently transferred to 5L G-Rex closed system for expansion until day 8. Vδ2 T cells enriched to >80% and were then subjected to cytokine stimulation process in the 5L G-Rex system to yield an s-Vδ2 T cell product for harvest. In most cases, the entire production process takes 8-14 days, depending on the particular donor. FIG. 9 schematically illustrates the process of Protocol 2.

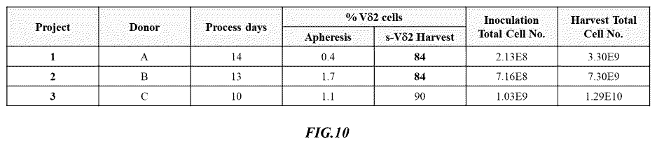

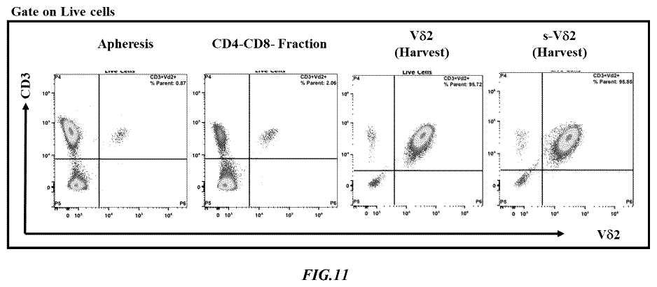

In this protocol, it should be appreciated that the enrichment of the Vδ2 T cells can be from about 1-6% of the CD 4/CD8-depleted T cell population to >80% at the day of harvest, and the table in FIG. 10 shows exemplary results for the enrichment process. As can be seen from the table, three independent studies were performed using blood material from different donors, providing a yield of between about 3-7 billion cells from 200-700 million cells. FIG. 11 depicts the corresponding results for flow dot plots. As can be readily taken from FIG. 11, the enrichment of live Vδ2 /s-Vδ2 T cells was substantial upon reaching harvest day.

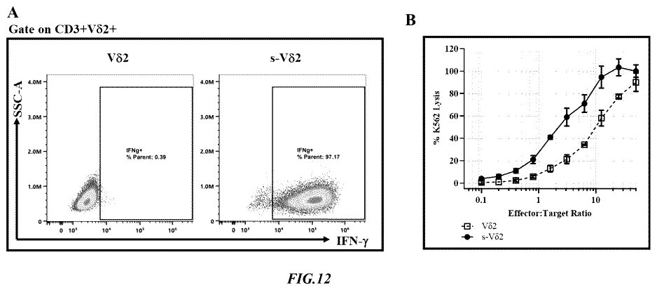

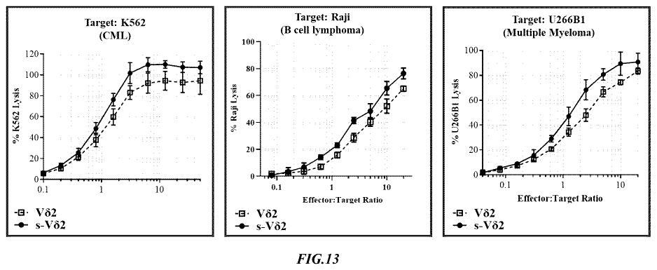

In a series of further experiments, the inventors characterized selected functional differences. Among other findings (not shown), the inventors noted that the s-Vδ2cells generated by CD4/CD8 depletion process showed significantly stronger potencies compared to Vδ2 cells, which is exemplarily shown in the results of FIG. 12 (A and B). Here, FIG. 12(A) shows flow dot plots for exemplary of IFNγ expression of Vδ2and s-Vδ2T cells harvest based on the CD4/CD8 depletion process and 5L G-Rex system. The s-Vδ2 T cells showed >95% IFN-γ expression while Vδ2 T cells only showed background signal. In terms of cytotoxic function, FIG. 12(B) and FIG. 13 show exemplary results for the evaluation of cytotoxicity of Vδ2and s-Vδ2 cells on K562 (CML), Raji (B cell lymphoma) and U266B1 (Multiple Myeloma) using a Calcein AM release assay. As can be readily taken from FIG. 12B and FIG. 13, s-Vδ2 showed significantly stronger potency in killing wide spectra of liquid tumor targets than Vδ2 cells.

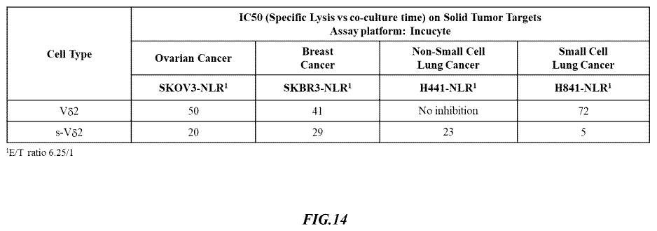

In yet another set of experiments to evaluate cytotoxicity, the inventors tested the Vδ2and s-Vδ2T cells against SKOV3-NLR, SKBR3-NLR, H441-NLR and H841-NLR cell lines with 10,000 cells per well for a period of 96 hours using an INCUCYTE™ (Live cell imaging and analysis system, Sartorius) platform. Exemplary results are depicted in FIG. 14. As can be readily seen, the s-Vδ2 T cells had once again substantial cytotoxicity against the target cells crossing various indications.

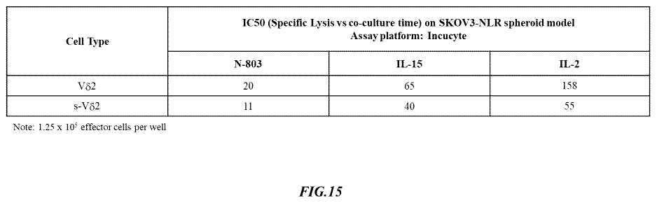

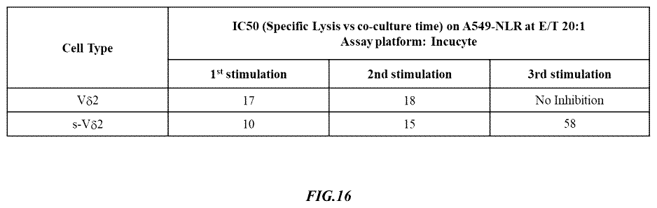

As showed in FIG. 15, compared with other common cytokines (e.g. IL2 and IL15), Vδ2 and s-Vδ2 T cells supplied with N-803 showed significantly stronger potency in the inhibition of SKOV3-NLR spheroid tumor model using INCUCYTE™ platform. And as shown in FIG. 16, s-Vδ2+ T cell showed superior persistence of sustained antitumor potency against A549-NLR cells based on the tumor rechallenge assays using INCUCYTE™: after three rounds of exposure (72 hours per round) to A549-NLR cells, s-Vδ2 T cells maintained high tumor inhibition capability while Vδ2 T cells lost tumor inhibition capability.

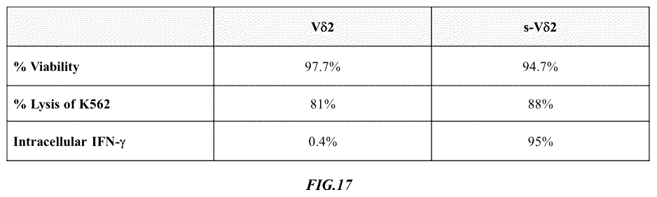

In addition, inventors demonstrated cryo-formulated Vδ2+ and s-Vδ2+ T cells maintained high anti-tumor potency and high viability after thawing as showed in FIG. 17.

### Further Experiments

CyTOF immunoprofiling and mRNA sequencing: CyTOF immunoprofiling is used to obtain deep immunoprofiling data and use bulk mRNA sequencing to obtain the transcriptomic information of Vδ2 /s-Vδ2 T cell products. This is valuable data for the following reasons: (A) Profile Vδ2 /s-Vδ2 T cell products to examine the cell components (αβT, NK, Vδ1, Vδ2, B, monocyte and so on). This data may help elucidate donor variations. (B) Compare the phenotypes of Vδ2 and s-Vδ2 T cell products on protein level and transcriptomic level.

In vivo efficacy of Vδ2 /s-Vδ2 T cell products: The inventors choose several tumor models to demonstrate the efficacy of Vδ2 /s-Vδ2 T cell products under different indications. NSG mice is used.

For un-engineered T cell products: (A) Liquid tumor: The inventors use K562 tumor model (luciferase expression). In brief, K562 expressing luciferase is inoculated in NSG mice via tail vein injection. After certain days, Vδ2 /s-Vδ2 T cells are dosed via tail vein injection. Tumor growth is measured according to luciferin bioluminescent signal. And mice blood, spleen and bone measure are analyzed using flow cytometry to track T cell expansion/distribution. (B) Solid tumor: Breast cancer SKBR3. In brief, SKBR3 tumor cells are inoculated into the mammary gland of female NSG mice. When the tumor is established, Vδ2 /s-Vδ2 T cells is dosed via tail vein injection. Then tumor growth is measured by size. Mice blood could be collected to track T cell expansion. At the end of the experiment, tumor and spleens are collected to analyze tumor infiltration and distribution. Additional solid tumor targets could be considered according to in vitro results.

For CAR-T cell products: The inventors use tumor models according to the targets of CARs. A Raji model for CD19-CAR. SKBR3 for HER2-CAR can be used. Exemplary CD19-CAR and HER2-CAR are disclosed in U.S. patent application Ser. No. 17/056,385, which is incorporated by reference herein.

ADCC of Vδ2 /s-Vδ2 drug products: γδ T cells express high level CD16 and are reported to show potent ADCC functional for cancer immunotherapy. The inventors collect additional data to demonstrate Vδ2 /s-Vδ2 products exhibit potent ADCC. This data support the high potencies of the Vδ2 /s-Vδ2 T cell products. In brief, the inventors test several antibodies on different target tumor cells. Different concentrations of antibodies are added to Vδ2 /s-Vδ2 T cells and target cells co-culture. Then cell cytotoxicity is evaluated using Calcein-release assay.

In some embodiments, the numbers expressing quantities of ingredients, properties such as concentration, reaction conditions, and so forth, used to describe and claim certain embodiments of the invention are to be understood as being modified in some instances by the term “about.” As used herein, the terms “about” and “approximately”, when referring to a specified, measurable value (such as a parameter, an amount, a temporal duration, and the like), is meant to encompass the specified value and variations of and from the specified value, such as variations of +/−10% or less, alternatively +/−5% or less, alternatively +/−1% or less, alternatively +/−0.1% or less of and from the specified value, insofar as such variations are appropriate to perform in the disclosed embodiments. Thus, the value to which the modifier “about” or “approximately” refers is itself also specifically disclosed. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

What is claimed is:

1. A method of expanding Vδ2 T cells from human apheresis material, the method comprising:
at least partially depleting the apheresis material of αβ T cells and/or CD4/CD8 T cells to thereby obtain a partially depleted T cell fraction enriched for Vδ2 T cells;
contacting the partially depleted T cell fraction with zoledronic acid and IL-15 for about 10-14 days to promote expansion of Vδ2 T cells, and
stimulating the expanded Vδ2 T cells with a cytokine cocktail comprised of Interleukin 12 (IL-12), Interleukin 15 (IL-15), and Interleukin 18 (IL-18) to generate memory-like Vδ2 T cells, and optionally cryopreserving the stimulated memory-like Vδ2 T cells.

2. The method of claim 1, wherein the IL-15 is present at a concentration of 64-84 ng/ml.

3. The method of claim 1, wherein the zoledronic acid is present at a concentration range of 2-10 μM, and optionally at a concentration of 5 μM.

4. The method of claim 1, wherein the cell media comprises X-VIVO™ 15 or T-VIVO.

5. The method of claim 1, wherein the cell media comprising the IL-15 is replenished every 3-4 days.

6. The method of claim 1, wherein the human apheresis material is CD4/CD8 T cell depleted.

7. The method of claim 1, wherein the memory-like Vδ2 T cells are comprised of effector memory (Vδ2 Tem) and central memory (Vδ2 Tcm) T cells.

8. The method of claim 1, wherein at least one of the Interleukin 12 (IL-12), Interleukin 15 (IL-15), and Interleukin 18 (IL-18) is a stabilized fusion protein derivative thereof.

9. The method of claim 1, wherein the memory-like Vδ2 T cells are comprised of effector memory (Vδ2 Tem) T cells.

10. The method of claim 1, wherein the memory-like Vδ2 T cells have high IFN-γ expression enhanced cytotoxicity against tumor cells.

11. The method of claim 1, wherein the Vδ2 and memory-like Vδ2 T cells show potent tumor inhibition potencies on a wide spectrum of liquid and solid tumors, crossing different indications, optionally wherein the Vδ2 and memory-like Vδ2 T cells maintain maximal tumor inhibition potency supplied with IL-15.

12. The method of claim 1, wherein the memory-like Vδ2 T cells show superior persistence of sustained antitumor potency, optionally wherein the cryo-formulated Vδ2 and memory-like Vδ2 T cells maintain high activity and rapid recovery after thawing.

13. The method of claim 1, wherein the IL-12 and IL-18 are added to the cytokine cocktail as a fusion protein or fusion protein complex comprised thereof.

14. The method of claim 1, wherein the Vδ2 T cells are enriched to comprise >90% of T cells after 10 days in culture, and optionally wherein the cytokine cocktail is added after 10 days in culture.

15. A pharmaceutical composition comprising human apheresis material, Zoledronic acid, and IL-15, wherein the human apheresis material is depleted of αβ T cells and/or CD4/CD8 T cells.

16. The composition of claim 15, wherein the IL-15 is present at a concentration of 64-84 ng/ml.

17. The composition of claim 15, wherein the zoledronic acid is present at a concentration of 5 μM.

18. The composition of claim 15, wherein the human apheresis material is CD4/CD8 T cell depleted.

19. A kit comprising human apheresis material, Zoledronic acid, IL-15 and directions for use thereof, wherein the human apheresis material is depleted of αβ T cells and/or CD4/CD8 T cells.

20. The kit of claim 19, wherein the IL-15 is present at a concentration of 64-84 ng/ml, and optionally wherein the zoledronic acid is present at a concentration of 5 μM.
